(19) United States
(12) Patent Application Publication
(10) Pub. No.: US 2024/0368101 A1
(43) Pub. Date: Nov. 7, 2024

(54) CONTINUOUS PRODUCTION SYSTEM FOR 2,5-FURANDICARBOXYLIC ACID AND CONTINUOUS PRODUCTION METHOD FOR 2,5-FURANDICARBOXYLIC ACID

(71) Applicant: Korea Institute of Science and Technology, Seoul (KR)

(72) Inventors: Dong Ki LEE, Seoul (KR); Byeong Cheul Moon, Seoul (KR); Jai Hyun Koh, Seoul (KR); Woong Hee Lee, Seoul (KR); Dahye Won, Seoul (KR); Ung Lee, Seoul (KR); Hyung-Suk Oh, Seoul (KR); Byoung Koun Min, Seoul (KR)

(21) Appl. No.: 18/379,709

(22) Filed: Oct. 13, 2023

(30) Foreign Application Priority Data
May 4, 2023 (KR) 10-2023-0058264

(51) Int. Cl. C07D 307/68

(52) U.S. Cl. CPC C07D 307/68

(57) ABSTRACT

Exemplary embodiments of the present invention may provide a continuous production system for 2,5-furandicarboxylic acid (FDCA), the continuous production system including: raw material supply units supplying an aqueous 5-hydroxymethylfurfural (HMF) solution and a basic aqueous solution, respectively; a micro-mixing unit mixing the aqueous HMF solution and the basic aqueous solution supplied from the raw material supply units, respectively, to form a raw material mixture; an electrochemical reaction unit synthesizing FDCA while passing the raw material mixture introduced from the micro-mixing unit in a single pass; and a product storage unit storing a product discharged from the electrochemical reaction unit.

### CROSS-REFERENCE TO RELATED APPLICATION

This application claims priority to and the benefit of Korean Patent Application No. 10-2023-0058264 filed in the Korean Intellectual Property Office on May 4, 2023, the entire contents of which are incorporated herein by reference.

### BACKGROUND OF THE INVENTION

### (a) Field of the Invention

Exemplary embodiments of the present invention relate to an electrochemical continuous production system capable of continuously producing 2,5-furandicarboxylic acid and a continuous production method of 2,5-furandicarboxylic acid, and more particularly, to a raw material separation and mixing continuous flow reaction system for continuously producing 2,5-furandicarboxylic acid and a continuous production method of high-purity 2,5-furandicarboxylic acid.

### (b) Description of the Related Art

Consumption of petroleum-derived plastics is continuously increasing, and carbon dioxide emissions for plastic production are approximately 1.7 gigatons, accounting for 15% of global carbon dioxide emissions. Therefore, as a substitute for the petroleum-derived plastics, a demand for CO₂-free plastics, which are largely composed of biomass-derived chemicals, is gradually increasing. Polyethylene furanoate (PEF) is one of the plastics based on vegetable raw materials and is attracting attention as a substitute for PET, which is produced from a petroleum-derived compound. Polyethylene furanoate (PEF) is one of the bio-based plastics that is receiving a lot of attention due to its high mechanical strength, excellent heat resistance, and excellent O₂ and CO₂ gas barrier properties. 2,5-Furandicarboxylic acid (FDCA), which is a monomer of PEF plastic, may be synthesized by an oxidation reaction of 5-hydroxymethylfurfural (HMF) produced by strong acid saccharification of cellulose.

In general, oxidation of HMF is performed under high temperature and pressure (to 140° C. and 40 bar) conditions using chemical oxidizers (02 and air) and noble metal catalysts (Pt, Pd, Au, and Ru). However, such a method according to the related art has a problem in that a large amount of energy is consumed. Recently, in accordance with an increase in proportion of renewable energy sources in power generation, a need to develop an electrochemical production method that may directly use electrical energy to be produced for a target chemical reaction without conversion of electricity into heat/pressure has increased.

To solve the above problems, the present applicant has applied for domestic patents KR 10-2021-0148900 and KR 10-2022-0128697 related to catalyst electrodes for efficient production of FDCA.

In a typical batch reaction system, the conversion reaction of HMF to FDCA is performed by injecting a strongly concentrated basic solution (4 to 10 M) into the reactor repeatedly to preserve the pH of the HMF solution above 14. This causes the degradation of HMF and its intermediates into useless polymeric compounds because the base-induced aldol condensation of carbonyl groups of HMF and its intermediates is accelerated by the high basicity of a solution Consequently, the reactant loss and impurity formation are inevitable in the batch reaction system, decreasing productivity and economic feasibility of FDCA production process.

### SUMMARY OF THE INVENTION

The present invention attempts to provide a continuous production system for 2,5-furandicarboxylic acid (FDCA) that may minimize a loss of materials due to degradation and may improve stability of the materials by shortening a reaction time, and a continuous production method for high-purity FDCA.

An exemplary embodiment of the present invention provides a continuous production system for FDCA, the continuous production system including: raw material supply units supplying an aqueous 5-hydroxymethylfurfural (HMF) solution and a basic aqueous solution, respectively; a micro-mixing unit mixing the aqueous HMF solution and the basic aqueous solution supplied from the raw material supply units, respectively, to form a raw material mixture; an electrochemical reaction unit synthesizing FDCA while passing the raw material mixture introduced from the micro-mixing unit in a single pass; and a product storage unit storing a product discharged from the electrochemical reaction unit.

The electrochemical reaction unit may include: a first electrode plate and a second electrode plate positioned to face each other; a membrane positioned between the first electrode plate and the second electrode plate; an anode and a cathode positioned to face each other on both sides of the membrane; an anode flow path positioned between the first electrode plate and the anode; and a cathode flow path positioned between the second electrode plate and the cathode.

Each of the anode flow path and the cathode flow path may be a lattice-type flow path in which a lattice is formed or a bulk-type flow path in which a lattice is not formed.

The lattice-type flow path may be a bar-type flow path in which a straight lattice is formed or a zigzag-type flow path in which a zigzag-shaped lattice is formed.

Another exemplary embodiment of the present invention provides a continuous production method for FDCA, the continuous production method including: supplying each of an aqueous HMF solution and a basic aqueous solution to a micro-mixing unit; mixing the supplied aqueous HMF solution and basic aqueous solution in the micro-mixing unit to form a raw material mixture; and supplying the raw material mixture to an electrochemical reaction unit and synthesizing FDCA while passing the raw material mixture through the electrochemical reaction unit in a single pass.

A time for the mixed aqueous HMF solution and basic aqueous solution to pass through the electrochemical reaction unit in a single pass after being supplied to the electrochemical reaction unit may be 1 minute to 5 minutes.

In the supplying of each of the aqueous HMF solution and the basic aqueous solution to the micro-mixing unit, a ratio of a flow rate of the aqueous HMF solution to a flow rate of the basic aqueous solution (aqueous HMF solution flow rate:basic aqueous solution flow rate) may be 1:0.5 to 1:2.

In the supplying of each of the aqueous HMF solution and the basic aqueous solution to the micro-mixing unit, the aqueous HMF solution and the basic aqueous solution may be supplied at a flow rate of 0.5 ml/min to 5 ml/min.

In the supplying of each of the aqueous HMF solution and the basic aqueous solution to the micro-mixing unit, a concentration of the aqueous HMF solution may be 0.5 wt % to 2.0 wt %.

In the mixing of the supplied aqueous HMF solution and basic aqueous solution in the micro-mixing unit to form the raw material mixture, a residence time of the aqueous HMF solution and the basic aqueous solution in the micro-mixing unit may be 0.5 seconds to 10 seconds.

The continuous production system for FDCA according to an exemplary embodiment of the present invention may minimize a loss of materials due to degradation, such that FDCA having a significantly low impurity content and high purity may be produced.

The continuous production system for FDCA according to an exemplary embodiment of the present invention supplies raw materials through a separate mixing method, such that chemical stability of materials during the reaction process may be secured, and high-purity FDCA may be produced.

The continuous production method for FDCA according to an exemplary embodiment of the present invention may shorten the reaction time and may continuously produce high-purity FDCA, such that the efficiency of the overall process may be improved.

### BRIEF DESCRIPTION OF THE DRAWINGS

FIG. 1 schematically illustrates a continuous production system for FDCA according to an exemplary embodiment of the present invention.

FIGS. 2A and 2B schematically illustrate structures of a micro-mixing unit according to an exemplary embodiment of the present invention.

FIG. 3 schematically illustrates an electrochemical reaction unit applied to a continuous flow reaction system for producing FDCA according to an exemplary embodiment of the present invention.

FIGS. 4A to 4C schematically illustrate electrode flow paths according to an exemplary embodiment of the present invention.

FIGS. 5A to 5C illustrate currents generated according to various types of combinations of a cathode flow path and an anode flow path and an applied voltage.

FIG. 6 illustrates a lab-scale continuous production system for FDCA.

FIG. 7 illustrates a change in current over a reaction time when the production system of FIG. 6 is used.

FIG. 8 schematically illustrates a production system for FDCA using an existing batch reactor.

FIG. 9 illustrates a change in current over a reaction time when the production system of FIG. 8 is used.

FIGS. 10A and 10B illustrate changes in concentrations of materials over time in Comparative Example 1.

### DETAILED DESCRIPTION OF THE EMBODIMENTS

Terminologies used herein are to mention only a specific exemplary embodiment, and are not to limit the present invention. Singular forms used herein include plural forms as long as phrases do not clearly indicate an opposite meaning. The term “comprising” used in the specification concretely indicates specific properties, regions, integers, steps, operations, elements, and/or components, and is not to exclude the presence or addition of other specific properties, regions, integers, steps, operations, elements, and/or components.

Unless defined otherwise, all terms including technical terms and scientific terms used herein have the same meanings as understood by those skilled in the art to which the present invention pertains. Terms defined in a generally used dictionary are additionally interpreted as having the meanings matched to the related technical document and the currently disclosed contents, and are not interpreted as ideal or very formal meanings unless otherwise defined.

The terms “first”, “second”, “third”, and the like are used to describe various parts, components, regions, layers, and/or sections, but are not limited thereto. These terms are only used to differentiate a specific part, component, region, layer, or section from another part, component, region, layer, or section. Accordingly, a first component, part, region, layer, or section which will be described hereinafter may be referred to as a second component, part, region, layer, or section without departing from the scope of the present invention.

In the description of the present invention, a reactant may be a raw material, a product, or a mixture thereof.

Hereinafter, exemplary embodiments of the present invention will be described in detail. However, these exemplary embodiments are provided as examples, and the present invention is not limited by these exemplary embodiments and is defined by only the scope of the claims to be described below.

An exemplary embodiment of the present invention may provide a continuous production system for FDCA.

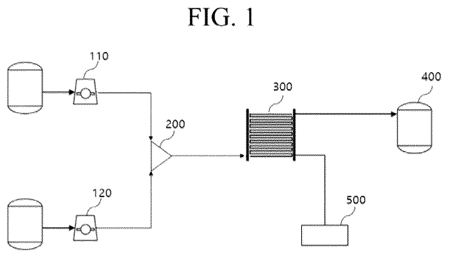

FIG. 1 schematically illustrates a continuous production system for FDCA according to an exemplary embodiment of the present invention.

Referring to FIG. 1, the continuous production system for FDCA according to an exemplary embodiment of the present invention may include: raw material supply units supplying an aqueous HMF solution and a basic aqueous solution, respectively; a micro-mixing unit 200 mixing the aqueous HMF solution and the basic aqueous solution supplied from the raw material supply units, respectively, to form a raw reactant mixture; an electrochemical reaction unit 300 synthesizing FDCA while passing the raw material mixture introduced from the micro-mixing unit 200 in a single pass; and a product storage unit 400 storing a product discharged from the electrochemical reaction unit 300.

First, the raw material supply unit may include an HMF supply unit 110 supplying an aqueous HMF solution and a base supply unit 120 supplying a basic aqueous solution. Each supply unit is a device that may continuously supply a raw material in a raw material storage tank to the micro-mixing unit 200 at the rear of the supply unit, and may be a peristaltic pump. The raw material supply unit is not particularly limited as long as it may continuously supply the raw material at a desired supply rate in the present invention. In the present invention, the HMF supply unit 110 and the base supply unit 120 may supply the aqueous HMF solution and the basic aqueous solution, respectively, at a supply rate of 0.5 ml/min to 5 ml/min. When the raw material supply rate of each of the HMF supply unit 110 and the base supply unit 120 is within the above range, a residence time range of the reactant in the electrochemical reaction unit targeted in the present invention may be satisfied, such that the degradation of HMF may be prevented or reduced, and a conversion rate of HMF and a yield of FDCA may be improved.

Although not illustrated in the drawings, storage tanks for storing the aqueous HMF solution and the basic aqueous solution, respectively, may be provided.

The continuous production system includes the micro-mixing unit 200 into which the aqueous HMF solution and the basic aqueous solution supplied from the HMF supply unit 110 and the base supply unit 120, respectively, are introduced.

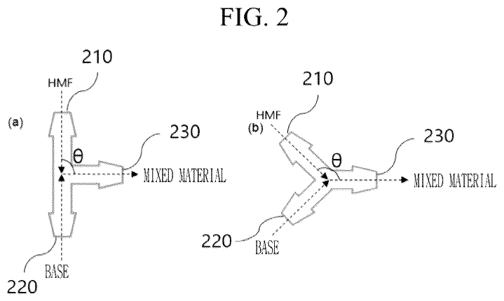

Referring to the structures of the micro-mixing unit 200 according to an exemplary embodiment of the present invention schematically illustrated in FIGS. 2A and 2B, the aqueous HMF solution and the basic aqueous solution are introduced into the micro-mixing unit 200 through an HMF inlet 210 and a base inlet 220, respectively, and are supplied to the electrochemical reaction unit 300 at the rear of the micro-mixing unit 200 through a mixture outlet 230. An angle (θ) between a flow direction of the aqueous HMF solution introduced through the HMF inlet 210 and a flow direction of the mixture discharged through the mixture outlet 230 may be 90° or more or less than 180°. Meanwhile, based on the flow direction of the mixture discharged through the mixture outlet 230, the aqueous HMF solution and the basic aqueous solution may be introduced at the same angle. This is advantageous in allowing the aqueous HMF solution and the basic aqueous solution to be introduced into the micro-mixing unit 200 to form a uniform mixture in a short time.

The electrochemical reaction unit 300 is positioned at the rear of the micro-mixing unit 200.

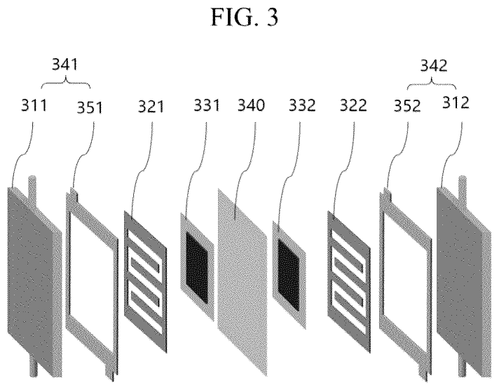

FIG. 3 schematically illustrates an electrochemical reaction unit applied to a continuous flow reaction system for producing FDCA according to an exemplary embodiment of the present invention. Referring to FIG. 3, the electrochemical reaction unit 300 according to an exemplary embodiment of the present invention includes: a first electrode plate 341 and a second electrode plate 342 positioned to face each other on both ends; a membrane 340 positioned between the first electrode plate 341 and the second electrode plate 342; an anode 331 and a cathode 332 positioned to face each other on both sides of the membrane 340; an anode flow path 321 positioned between the first electrode plate 341 and the anode 331; and a cathode flow path 322 positioned between the second electrode plate 342 and the cathode 332.

The first electrode plate 341 and the second electrode plate 342 are conductive electrode plates that serve to pass a current for an electrochemical reaction, include gold-coated brass electrode plates 351 and 352 and end plates 311 and 312, which are metal housings formed of nickel-coated stainless steel (SUS316L) provided to protect the brass electrode plates 351 and 352, respectively, and may contain a catalyst, as necessary. The material thereof is not particularly limited as long as it may allow a desired current to pass in the present invention. Meanwhile, the first electrode plate 341 and the second electrode plate 342 adjust an electric voltage and current applied by a power supply unit 500.

The membrane 340 is an ion conductive membrane, and an anionic conductive membrane or a cationic conductive membrane may be used.

The anode 331 and the cathode 332 may be metal electrodes containing one or more selected from nickel, copper, and other metal catalysts, and may have an area of 1 cm² to 150 cm², and specifically, 5 cm² to 100 cm².

The anode flow path 321 positioned between the first electrode plate 341 and the anode 331 may provide a path through which the raw material mixture formed by mixing the aqueous HMF solution and the basic aqueous solution supplied from the micro-mixing unit 200 passes. The raw material mixture may produce FDCA through an electrochemical reaction while passing through the anode flow path 321, and the FDCA discharged from the electrochemical reaction unit 300 may be stored in the product storage unit 400.

The cathode flow path 322 positioned between the second electrode plate 342 and the cathode 332 may provide a path through which water, humidified air, or the basic aqueous solution passes, and water may electrolyzed while passing through the cathode flow path 322 to produce hydrogen.

An electrode flow path 320, that is, the anode flow path 321 and the cathode flow path 322 may be a plate with a hole formed in the center. The water or raw material mixture may perform an electrochemical reaction while passing through the hole. In this case, the plate is a path for transmitting a current to the anode 331 and the cathode 332. A thickness of the plate may be 0.5 cm to 1 cm to minimize electrical resistance, and a material of the plate may be one or more selected from nickel and titanium.

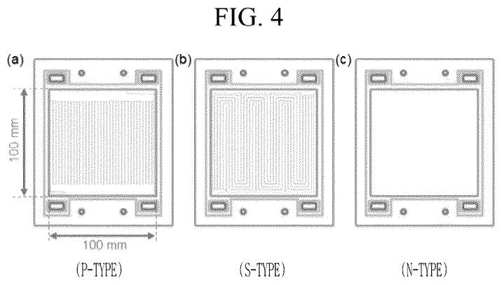

Meanwhile, one or more lattices may be formed in the hole. Specifically, the reactant may be separated by the lattices and pass through the electrode flow path 320. Referring to the schematic views of the electrode flow path 320 according to an exemplary embodiment of the present invention illustrated in FIGS. 4A to 4C, FIGS. 4A and 4B schematically illustrate lattice-type flow paths in which a lattice is formed in the hole, and FIG. 4C illustrates an N-type flow path that is a bulk (none)-type flow path in which a lattice is not formed. Specifically, FIG. 4A illustrates a P-type flow path that is a bar (parallel)-type electrode flow path in which a bar-shaped lattice is formed, and FIG. 4B illustrates an S-type flow path that is a zigzag (serpentine)-type electrode flow path in which a zigzag-shaped lattice is formed.

The anode flow path 321 and the cathode flow path 322 may be used in selective combination of the lattice-type flow path and the bulk-type flow path. Specifically, the cathode flow path 322 may be a bulk-type flow path, and the anode flow path 321 may be a bar-type flow path.

A lattice spacing may be 2 mm to 5 mm. When the lattice spacing is within the above range, the reactant may pass smoothly and the flow rate of the aqueous solution may be easily controlled.

Another exemplary embodiment of the present invention provides a continuous production method FDCA, the continuous production method including: supplying each of an aqueous HMF solution and a basic aqueous solution to a micro-mixing unit; mixing the supplied aqueous HMF solution and basic aqueous solution in the micro-mixing unit to form a raw material mixture; and supplying the raw material mixture to an electrochemical reaction unit and synthesizing FDCA while passing the raw material mixture through the electrochemical reaction unit in a single pass.

The aqueous HMF solution may be prepared and stored in a separate container. Meanwhile, the aqueous HMF solution may be an aqueous solution at a concentration of 0.5 wt % to 5.0 wt %, specifically, an aqueous solution at a concentration of 0.5 wt % to 2.0 wt %, and more specifically, an aqueous solution at a concentration of 0.5 wt % to 1.2 wt %. In this case, a pH value may be around 7.

The basic aqueous solution may be prepared and stored in a separate container. The basic aqueous solution may be one or more selected from an aqueous potassium hydroxide (KOH) solution, an aqueous sodium hydroxide (NaOH) solution, an aqueous lithium hydroxide (LiOH) solution, and an aqueous cesium hydroxide (CsOH) solution, and a pH value of the basic aqueous solution may be around 14.

The aqueous HMF solution and the basic aqueous solution may be continuously supplied by a pump and may be supplied at a supply rate of 0.5 ml/min to 5.0 ml/min. Meanwhile, the aqueous HMF solution and the basic aqueous solution may be supplied at the same supply rate. This is advantageous in that the aqueous HMF solution and the basic aqueous solution are uniformly mixed in the micro-mixing unit in a short time.

The aqueous HMF solution and the basic aqueous solution are respectively supplied to the micro-mixing unit and then mixed to form a raw material mixture. A ratio of a flow rate of the aqueous HMF solution to a flow rate of the basic aqueous solution (aqueous HMF solution flow rate:basic aqueous solution flow rate) may be 1:0.5 to 1:2, and specifically, 1:1. Meanwhile, a residence time of the raw material mixture in the micro-mixing unit may be 0.5 seconds to 10 seconds. When the residence time is within the above range, the aqueous HMF solution and the basic aqueous solution are uniformly mixed, which is advantageous in reducing the overall process time.

The raw material mixture formed in the micro-mixing unit may be introduced into the electrochemical reaction unit at a flow rate of 1 ml/min to 10 ml/min. When the flow rate of the raw material mixture is within the above range, it is advantageous for improving the conversion rate of HMF and the yield of FDCA.

As the raw material mixture passes through the electrochemical reaction unit, an electrochemical reaction occurs and HMF is converted into FDCA. In this case, a time for the raw material mixture to pass through the electrochemical reaction unit may be 0.5 minutes to 5 minutes, and specifically, 1 minute to 4 minutes. When the time for the raw material mixture to pass through the electrochemical reaction unit, that is, the residence time in the electrochemical reaction unit is within the above range, the degradation of HMF is minimized, which is advantageous for improving the yield of FDCA. When the residence time in the electrochemical reaction unit is shorter than 1 minute, the conversion yield of HMF to FDCA is low, and when the residence time in the electrochemical reaction unit is longer than 5 minutes, generation of impurities increases due to degradation of HMF.

Meanwhile, an output of power applied to the electrochemical reaction unit may be 5 W to 60 W. Specifically, an applied voltage may be 2.0 V to 3.0 V, and more specifically, 2.0 V to 2.6 V. In addition, a current may be 5 A to 20 A, and specifically, 6.5 A to 20 A.

Hereinafter, Examples of the present invention will be described in detail. However, these Examples are provided as examples, and the present invention is not limited by these Examples and is defined by only the scope of the claims to be described below.

### Experimental Example 1: Comparison of Currents Generated According to Flow Path Combination for Each Type

An anode flow path 321 and a cathode flow path 322 were arbitrarily combined into one of the N-type, P-type, and S-type flow paths, and 2,5-furandicarboxylic acid production currents of an electrochemical reactor according to an applied voltage were compared.

Specifically, a voltage was applied in a range of 1.6 V to 2.4 V using a constant voltage test method, and a current value was measured for 5 to 10 minutes.

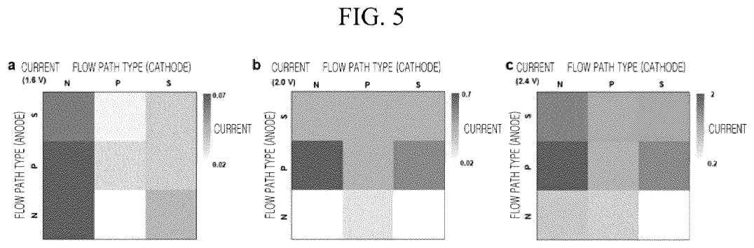

FIG. 5 illustrates currents generated according to various types of combinations of the anode flow path 321 and the cathode flow path 322 and the applied voltage. Referring to FIG. 5, when the cathode flow path (anode flow path of FIG. 5) was an N-type flow path and the anode flow path (cathode flow path of FIG. 5) was a P-type flow path, it was shown that the generated current was high regardless of a change in applied voltage.

### Experimental Example 2: FDCA Continuous Production Experiment

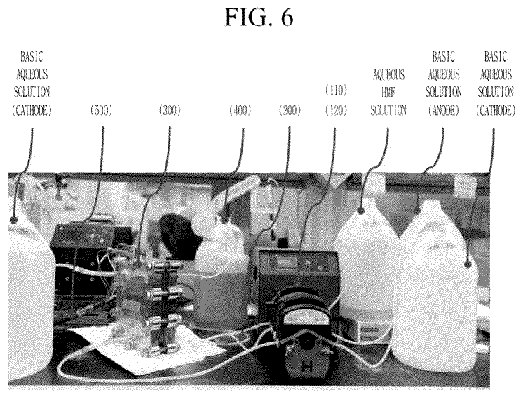

FIG. 6 illustrates a lab-scale continuous production system for FDCA.

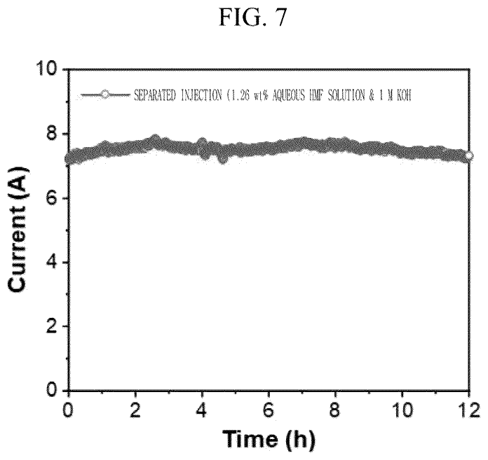

When an FDCA conversion experiment was performed using the continuous production system for FDCA of FIG. 6 and using a 1.26 wt % aqueous HMF solution and a 1 M aqueous KOH solution as raw materials, a change in current over a reaction time was observed and summarized in FIG. 7.

Referring to FIG. 7, it was confirmed that the aqueous HMF solution and the aqueous KOH solution were mixed separately, a reactant at a constant concentration was continuously supplied to the continuous flow reaction device according to the present invention, that is, the electrochemical reaction unit, and an FDCA production current was maintained at an almost constant level without a significant change over time.

### Comparative Example 1: Production of FDCA Using Batch Reactor

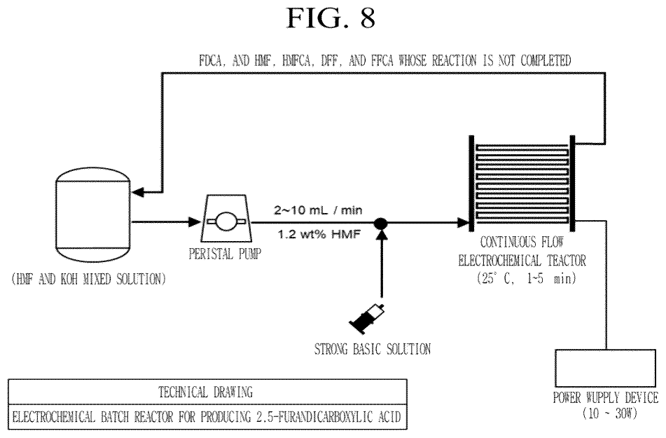

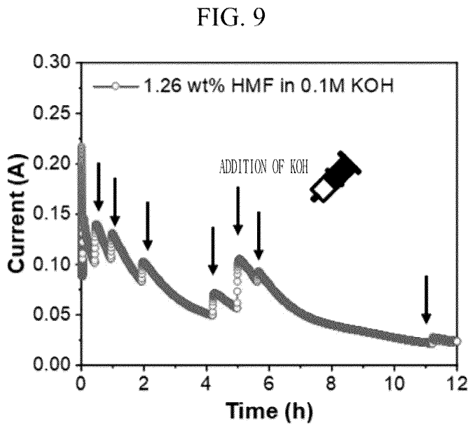

When an FDCA conversion experiment was performed using the production system for FDCA using a batch reactor illustrated in FIG. 8 and using a 1.26 wt % aqueous HMF solution and a 4 M aqueous KOH solution as raw materials, a change in current over time was observed. The result is illustrated in FIG. 9.

Referring to FIG. 9, the current decreased over the reaction time in the batch reactor, which was due to a decrease in reactant contained in the batch reactor. In addition, it was confirmed that the current fluctuated according to addition of KOH to solve the pH decrease problem, and it was considered that impurities were generated during this process due to spontaneous degradation of the reactant due to the high pH of 4 M KOH.

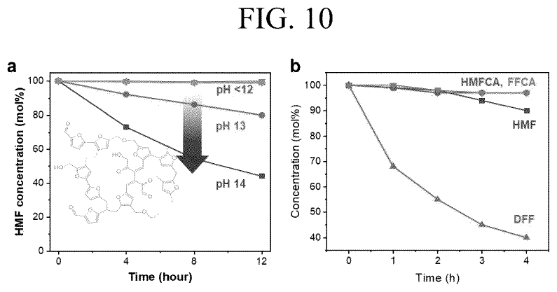

In addition, when the pH was set to 7, 9, 12, 13, and 14 in Comparative Example 1, changes in concentration of HMF and concentrations of various intermediate products in the aqueous solution over time were illustrated in FIGS. 10A and 10B.

Referring to FIG. 10A, it was confirmed that as the pH increased, the decrease in concentration of HMF in the aqueous solution increased over time, and when the pH was 12 or lower, no decrease in concentration was observed.

FIG. 10B illustrates the changes in concentrations of various intermediate products over time at pH 13. Referring to FIG. 10B, not only HMF as a reaction raw material, but also the intermediate products produced during the reaction were unstable in the same basic environment. In particular, diformylfuran (DFF) was degraded nearly 6 times faster than HMF, and this degradation led to formation of polymeric impurities.

Table 1 shows the results of the HMD degradation rate and the FDCA yield over the reaction time at pH 13 in Comparative Example 1.

TABLE 1

| Reaction | | HMF degradation rate | Theoretical FDCA maximum |
|---|---|---|---|
| time | | (%) | yield (%) |
| 0 | | 0 | 100 |
| 10 | min | 0.3 | 99.7 |
| 30 | min | 1 | 99 |
| 1 | hour | 2 | 98 |
| 5 | hour | 10 | 90 |
| 10 | hour | 20 | 80 |

Referring to Table 1, it is considered that the HMF degradation rate increases and the theoretical FDCA yield decreases over the reaction time.

The HMF degradation rate was calculated as HMF degradation rate (%)=((C_{O}−Cₜ)/C_{O})×100 using the initial concentration (C_{O}) of HMF contained in the aqueous solution with pH 13 and the concentration (Cₜ) of HMF after time (t). The theoretical FDCA maximum yield was calculated as theoretical FDCA maximum yield (%)=C/C_{O}×100 using the concentration (Cₜ) of residual HMF remaining in the aqueous solution without degradation.

Meanwhile, the HMF conversion rate (%) was a ratio of the concentration (C_{O}−Cₜ) of HMF consumed after the reaction process to the initial concentration (C_{O}) of HMF in the electrochemical reactor, and was calculated as ((C_{O}−Cₜ)/C_{O})×100. The FDCA yield (%) was a ratio of the concentration (C_{FDCA}) of FDCA produced after the reaction to the initial concentration (C₀) of HMF, and was calculated as (C_{FDCA}/C₀)×100.

### Experimental Example: Production of FDCA Using Continuous Reactor of Present Invention

An experiment was performed for 40 hours or longer using the lab-scale continuous production system for FDCA of FIG. 6 and using the 1.26 wt % aqueous HMF solution and the 1 M aqueous KOH solution as raw materials.

The FDCA conversion reaction was performed by supplying each of the 1.26 wt % aqueous HMF solution and the 1 M aqueous KOH solution and mixing the aqueous solutions in a micro-mixing unit and then supplying the mixture to the electrochemical reaction unit while passing the mixture.

The product discharged from the electrochemical reaction unit was collected and analyzed using High-Performance Liquid Chromatography, and the HMF conversion rate and the FDCA yield were calculated using the methods described above.

The conditions and results of each experiment using a reactor with an anode and a cathode each having a size of 100 cm² were summarized in Table 2.

TABLE 2

| | | | | | | | HMF | |
|---|---|---|---|---|---|---|---|---|
| | HMF | KOH | Applied | | | Residence | conversion | |
| | concentration | concentration | voltage | Current | Flow rate | time | rate | FDCA yield |
| No. | (wt %) | (M) | (V) | (A) | (mL/min) | (min) | (%) | (%) |
| Example 1 | 1.2 | 1.0 | 2.0 | 7.0 | 1.0 | 2.0 | 99.0 | 95.8 |
| Example 2 | 1.2 | 1.0 | 2.2 | 10.0 | 1.0 | 2.0 | 99.8 | 99.3 |
| Example 3 | 1.2 | 1.0 | 2.2 | 10.0 | 2.0 | 1.0 | 99.4 | 97.0 |
| Example 4 | 1.2 | 1.0 | 2.4 | 10.0 | 0.5 | 4.0 | 99.9 | 99.2 |
| Example 5 | 0.6 | 1.0 | 2.2 | 6.5 | 1.0 | 2.0 | 99.6 | 98.0 |
| Example 6 | 1.2 | 1.0 | 2.6 | 20.0 | 1.0 | 2.0 | 99.6 | 97.8 |
| Comparative | 0.2 | 1.0 | 2.0 | 4.7 | 2.0 | 1.0 | 91.7 | 84.1 |
| Example 1 | | | | | | | | |
| Comparative | 0.6 | 1.0 | 2.0 | 2.0 | 1.0 | 2.0 | 98.8 | 90.2 |
| Example 2 | | | | | | | | |
| Comparative | 0.6 | 1.0 | 2.0 | 2.0 | 2.0 | 1.0 | 87.1 | 79.0 |
| Example 3 | | | | | | | | |
| Comparative | 1.2 | 1.0 | 1.6 | 0.5 | 1.0 | 2.0 | 98.7 | 92.6 |
| Example 5 | | | | | | | | |
| Comparative | 2.5 | 1.0 | 2.0 | 3.5 | 1.0 | 2.0 | 99.3 | 93.5 |
| Example 6 | | | | | | | | |
| Comparative | 2.5 | 1.0 | 2.2 | 3.6 | 1.0 | 2.0 | 99.1 | 83.7 |
| Example 7 | | | | | | | | |
| Comparative | 2.5 | 1.0 | 2.4 | 3.8 | 1.0 | 2.0 | 97.3 | 72.7 |
| Example 8 | | | | | | | | |
| Comparative | 1.2 | 1.0 | 2.2 | 10.0 | 4.0 | 0.5 | 98.0 | 95.3 |
| Example 9 | | | | | | | | |
| Comparative | 1.2 | 1.0 | 2.2 | 10.0 | 10 | 0.2 | 85.2 | 72.7 |
| Example 10 | | | | | | | | |
| Comparative | 1.2 | 1.0 | 2.2 | 10.0 | 20 | 0.1 | 65.5 | 53.3 |
| Example 11 | | | | | | | | |
| Comparative | 1.2 | 1.0 | 2.2 | 10.0 | 40 | 0.05 | 56.7 | 42.1 |
| Example 12 | | | | | | | | |

Referring to Table 2, in the cases of Examples 1 to 6 in which the residence time of the reactant in the electrochemical reaction unit was 1 minute to 5 minutes, the concentration of HMF as a raw material was 0.5 wt % to 2.0 wt %, and the applied power output was 10 W to 60 W, it was confirmed that the conversion rate of HMF as a raw material was 99% or more and the FDCA yield was 95% or more.

The present invention is not limited to the exemplary embodiments, but may be prepared in various different forms, and it will be apparent to those skilled in the art to which the present invention pertains that the exemplary embodiments may be implemented in other specific forms without departing from the spirit or essential feature of the present invention. Therefore, it is to be understood that the exemplary embodiments described hereinabove are illustrative rather than restrictive in all aspects.

### DESCRIPTION OF SYMBOLS

100: Raw material supply unit
110: HMF supply unit
120: Base supply unit
200: Micro-mixing unit
300: Electrochemical reaction unit
400: Storage unit
500: Power supply unit
331: Anode
332: Cathode
320: Electrode flow path
321: Anode flow path
322: Cathode flow path
340: Membrane
341: First electrode plate
342: Second electrode plate
311, 312: End plate
351, 352: Brass electrode plate

What is claimed is:

1. A continuous production system for 2,5-furandicarboxylic acid (FDCA), comprising:
raw material supply units supplying an aqueous 5-hydroxymethylfurfural (HMF) solution and a basic aqueous solution, respectively;
a micro-mixing unit mixing the aqueous HMF solution and the basic aqueous solution supplied from the raw material supply units, respectively, to form a raw material mixture;
an electrochemical reaction unit synthesizing FDCA while passing the raw material mixture introduced from the micro-mixing unit in a single pass; and
a product storage unit storing a product discharged from the electrochemical reaction unit.

2. The continuous production system of claim 1, wherein:
the electrochemical reaction unit includes:
a first electrode plate and a second electrode plate positioned to face each other;
a membrane positioned between the first electrode plate and the second electrode plate;
an anode and a cathode positioned to face each other on both sides of the membrane;
an anode flow path positioned between the first electrode plate and the anode; and
a cathode flow path positioned between the second electrode plate and the cathode.

3. The continuous production system of claim 2, wherein:
each of the anode flow path and the cathode flow path is a lattice-type flow path in which a lattice is formed or a bulk-type flow path in which a lattice is not formed.

4. The continuous production system of claim 3, wherein:
the lattice-type flow path is a bar-type flow path in which a straight lattice is formed or a zigzag-type flow path in which a zigzag-shaped lattice is formed.

5. A continuous production method for FDCA, comprising:
supplying each of an aqueous HMF solution and a basic aqueous solution to a micro-mixing unit;
mixing the supplied aqueous HMF solution and basic aqueous solution in the micro-mixing unit to form a raw material mixture; and
supplying the raw material mixture to an electrochemical reaction unit and synthesizing FDCA while passing the raw material mixture through the electrochemical reaction unit in a single pass.

6. The continuous production method of claim 5, wherein:
a time for the mixed aqueous HMF solution and basic aqueous solution to pass through the electrochemical reaction unit in a single pass after being supplied to the electrochemical reaction unit is 1 minute to 5 minutes.

7. The continuous production method of claim 5, wherein:
in the supplying of each of the aqueous HMF solution and the basic aqueous solution to the micro-mixing unit,
a ratio of a flow rate of the aqueous HMF solution to a flow rate of the basic aqueous solution (aqueous HMF solution flow rate:basic aqueous solution flow rate) is 1:0.5 to 1:2.

8. The continuous production method of claim 5, wherein:
in the supplying of each of the aqueous HMF solution and the basic aqueous solution to the micro-mixing unit,
the aqueous HMF solution and the basic aqueous solution are supplied at a flow rate of 0.5 ml/min to 5 ml/min.

9. The continuous production method of claim 5, wherein:
in the supplying of each of the aqueous HMF solution and the basic aqueous solution to the micro-mixing unit,
a concentration of the aqueous HMF solution is 0.5 wt % to 2.0 wt %.

10. The continuous production method of claim 5, wherein:
in the mixing of the supplied aqueous HMF solution and basic aqueous solution in the micro-mixing unit to form the raw material mixture,
a residence time of the aqueous HMF solution and the basic aqueous solution in the micro-mixing unit is 0.5 seconds to 10 seconds.
